(19) United States
(12) Patent Application Publication
(10) Pub. No.: US 2004/0110973 A1
(43) Pub. Date: Jun. 10, 2004

(54) Olefin oxide catalysts

(76) Inventor: Marek Matusz, Houston, TX (US)

Correspondence Address: Jennifer D. Adamson, Shell Oil Company, Intellectual Property Services, P. O. Box 2463, Houston, TX, 77252-2463, US

(21) Appl. No.: 10/695,357

(22) Filed: Oct. 28, 2003

Related U.S. Application Data
(60) Provisional application No. 60/421,729, filed on Oct. 28, 2002.

(51) Int. Cl. C07D 301/10

(52) U.S. Cl. 549/534

(57) ABSTRACT

The invention provides a process for the oxidation of olefins having three or more carbon atoms in which the olefin is reacted with oxygen in the presence of a catalyst containing silver and a promoter containing potassium and a promoter containing rhenium deposited on an α-alumina carrier, in which the potassium promoter provides potassium at a concentration of up to 120 μmole per gram of catalyst. The invention further provides a catalyst composition for the oxidation of olefins having three or more carbon atoms in which the catalyst contains silver and a promoter containing potassium and a promoter containing rhenium deposited on an α-alumina carrier, in which the potassium promoter provides potassium at a concentration of from 8 μmole per gram to 120 μmole per gram of catalyst.

### FIELD OF THE INVENTION

The present invention relates to silver-containing supported catalysts having a promoter, processes for their preparation, and their use for preparing olefin oxides especially propylene oxide. The invention also relates to a process for making derivatives of olefins.

### BACKGROUND OF THE INVENTION

The direct oxidation of olefins to an olefin oxide by molecular oxygen is a method currently used for commercial production of ethylene oxide. The typical catalyst for such purpose contains metallic or ionic silver, optionally modified with various promoters and activators. Most such catalysts contain a porous, inert support or carrier upon which the silver and promoters are deposited.

In olefin oxidations, catalyst performance may be assessed on the basis of selectivity, activity and stability of operation. The selectivity is the mole percentage of the desired olefin oxide produced relative to the amount of olefin consumed. As the catalyst ages, the percentage of the olefin reacted normally decreases with time and to maintain a constant level of olefin oxide production the temperature of the reaction is increased. However, this adversely affects the selectivity of the conversion to the desired olefin oxide. Because the reactor equipment can withstand temperatures only up to a certain level, it is necessary to terminate the reaction when the temperature reaches an unacceptable level. Thus, the longer the selectivity can be maintained at a high level and the oxidation can be performed at an acceptable temperature, the longer the catalyst charge can be kept in the reactor and the more product is obtained. Quite modest improvements in the maintenance of selectivity over long periods yields huge dividends in terms of process efficiency.

It is also known, that the catalysts and reaction conditions which are best suited for ethylene oxide production do not give comparable results in the direct oxidation of higher olefins such as propylene. The discovery of processes capable of providing propylene oxide, and oxides of higher olefins, by vapor phase direct oxidation in higher yields and selectivities than are presently attainable thus would be most desirable.

### SUMMARY OF THE INVENTION

The invention provides a process for the oxidation of an olefin having three or more carbon atoms, which process comprises reacting the olefin with oxygen in the presence of a catalyst composition comprising silver, a promoter comprising potassium and a promoter comprising rhenium deposited on an α-alumina carrier, wherein said potassium promoter provides potassium at a concentration of up to 120 μmole per gram of catalyst composition.

Additionally, the invention provides a catalyst composition comprising silver, a promoter comprising potassium and a promoter comprising rhenium deposited on an α-alumina carrier, wherein said potassium promoter provides potassium at a concentration of from 8 μmole to 120 μmole per gram of catalyst composition.

### BRIEF DESCRIPTION OF THE FIGURES

FIG. 1 illustrates the performance of a potassium promoted silver catalyst as measured by selectivity and oxygen conversion.

FIG. 2 shows the work rate for a potassium promoted silver catalyst.

FIG. 3 illustrates the performance of a rhenium containing potassium promoted silver catalyst as measured by selectivity and oxygen conversion.

FIG. 4 shows the work rate for a rhenium containing potassium promoted silver catalyst.

### DETAILED DESCRIPTION OF THE INVENTION

The present invention provides a catalyst composition and a process for the oxidation of an olefin comprising three or more carbon atoms. In a preferred embodiment, the process comprises reacting the olefin with oxygen in the presence of a catalyst composition comprising silver, a potassium promoter and a rhenium promoter deposited on a carrier, wherein the potassium promoter provides potassium at a concentration of from 5-200 μmole, more preferably 12-100 μmole per gram of catalyst composition and the rhenium metal promoter provides rhenium at a concentration of from 1-30 μmole per gram of catalyst composition.

The invention is further directed to a process for making olefin oxide derivatives. such as olefin glycol, polylalkylene oxide, etc. from the olefin oxide made from the instant process. Any process known to one skilled in the art for converting olefin oxide to olefin oxide derivatives can be utilized for converting the olefin oxide. As one specific embodiment of the present invention, propylene oxide derivatives, such as polypropylene oxide, propylene glycol, are made from the propylene oxide made from the present invention, using suitable processes known to one skilled in the art.

The term “improved catalyst performance” means that there is an improvement in at least one of the catalyst properties, which catalyst properties include catalyst activity, selectivity, activity or selectivity performance over time, operability (i.e. resistance to run-away), conversion, and work rate.

In accordance with this invention, the addition of a promoter comprising rhenium, for example, ammonium perrhenate (NH₄ReO₄), to potassium promoted catalysts improves catalyst selectivity and productivity in the oxidation of olefins comprising at least three carbon atoms. The catalysts of the invention are particularly useful for preparing propylene oxide via silver catalyzed oxidation of propylene.

The quantity of silver supported on the carrier may be selected within wide ranges. Suitably the quantity of silver is in the range of from 0.5% by weight to 60% by weight, more preferably from 0.75% by weight to 58% by weight, and most preferably from 1% by weight to 55% by weight, relative to the total weight of the catalyst composition.

The quantity of potassium in the catalyst is typically at least 5 μmol, preferably from 8 μmol to 120 μmol per gram of catalyst, and more preferably from 12 μmol to 100 μmol per gram of catalyst.

The quantity of rhenium in the catalyst is typically from 1 μmol to 20 or 30 μmol per gram of catalyst, more preferably from 2 μmol to 25 μmol per gram of catalyst, and most preferably from 3 μmol to 20 μmol per gram of catalyst.

The catalyst carrier is based on an α-alumina. The α-alumina material may be a natural or artificial material and it may contain as additional components, refractory materials, silicon carbide, clays, zeolites, charcoal and alkaline earth metal carbonates, for example calcium carbonate. Refractory materials that can be used include alumina other than α-alumina, magnesia, zirconia and silica.

Typically, the carrier is a porous carrier, preferably having a specific surface area of from 0.01 m²/g to 50 m²/g, in particular from 0.05 m²/g to 30 m²/g, as measured by the B.E.T. method, and an apparent porosity of from 0.05 ml/g to 3 ml/g, in particular from 0.1 ml/g to 2 ml/g, as measured by conventional water absorption technique. The B.E.T. method as referred to herein has been described in detail in S. Brunauer, P. Y. Emmett and E. Teller, J. Am. Chem. Soc. 60, 309-16 (1938).

Of particular interest are α-aluminas which have a specific surface area of from 0.1 m²/g to 25 m²/g, preferably from 0.3 m²/g to 10 m²/g, more preferably from 1 m²/g to 5 m²/g, as measured by the B.E.T. method, and which have an apparent porosity of from 0.1 ml/g to 1.2 ml/g, in particular from 0.1 ml/g to 0.8 ml/g, as measured by conventional water absorption technique. Preferably, these α-aluminas have a relatively uniform pore diameter. Specific examples of such α-aluminas are marketed by NorPro under the trademark ALUNDUM® and by Südchemie.

Other α-alumina carriers which can be used have a surface area of at least 0.7 m²/g, and a pore size distribution such that pores with diameters in the range of from 0.2 μm to 10 μm represent at least 70% of the total pore volume and such pores together provide a pore volume of at least 0.2 ml/g, relative to the weight of the carrier.

Additional α-alumina carriers that can be used for supporting the catalysts of the invention are made from mixtures comprising: (a) from 50% w to 90% w of a first particulate α-alumina having an average particle size (d₅₀) of from more than 10 μm up to 100 μm, preferably from 11 μm to 60 μm, more preferably from 12 μm to 40 μm; and (b) from 10% w to 50% w of a second particulate α-alumina having a d₅₀ of from 1 μm to 10 μm, preferably from 2 μm to 6 μm; the % w being based on the total weight of α-alumina in the mixture. The mixture is then fired to form the carrier. In one embodiment, amongst others, the mixture may be formed into shaped bodies and then the shaped bodies are fired to form the carrier. When the shaped bodies are formed by extrusion, it may be desirable to include conventional burnout materials and/or extrusion aids, and an aqueous liquid, e.g. water, in the mixture.

The α-alumina particles may be commercially available, or they may readily be made, for example, by subjecting course materials to grinding and sieving operations. In an embodiment of the present invention, the smaller particles may be prepared from the larger particles by grinding, and the ground and un-ground particles may then be combined. In another embodiment of the present invention, the desired mixture of large and small particles may be formed by grinding relatively large particles to the extent that the mixture of particles has the desired bimodal particle size distribution.

When making α-alumina carriers which are mixtures of different types of α-alumina, typically, the first particulate α-alumina is employed in a quantity of from 65% w to 75% w, relative to the total weight of α-alumina in the mixture. Typically, the second particulate α-alumina is employed in a quantity of from 25% w to 35% w, relative to the total weight of α-alumina in the mixture.

Because the carrier is an α-alumina carrier, more in particular comprising at least 60% w, at least 80% w, at least 90% w, at least 95% w or at least 99.5% w α-alumina, it is preferred that a coating material based on a silica-containing composition comprising a crystallization inhibitor is included, thus inhibiting the formation of crystalline silica-containing compositions. It is preferred that this material provides a coating of a non-crystalline silica compound on the carrier surface. Preferably, the coating material also acts as a bond material for the α-alumina carrier.

Typically, silica-containing compositions for use as a coating material comprise an amorphous silica compound which may be, for example, a silica sol, a precipitated silica, an amorphous silica, or an amorphous alkali metal silicate or aluminasilicate. Typically, silica-containing compositions for use as a coating material may also comprise hydrated alumina. The crystallization inhibitor that is most conveniently incorporated is an alkali metal compound, in particular a water soluble salt, such as a sodium or potassium salt.

A convenient coating material may comprise a mixture of boehmite, ammonium silicate or silica sol, and a water soluble sodium salt. Similar effects can be achieved by incorporation of conventional ceramic bonds formulated to contain aluminosilicates and an alkali metal component.

Because the carrier is an α-alumina carrier, more in particular comprising at least 60% w, at least 80% w, at least 90% w, at least 95% w or at least 99.5% w α-alumina, it is preferred that the coating material is based on (a) from 1% w to 10% w, in particular 2% w to 5% w, of an alumina hydrate, calculated as aluminum oxide relative to the weight of the α-alumina; (b) from 0.1% w to 10% w, in particular 0.2% w to 5% w, of an amorphous silica compound, as specified hereinbefore, calculated as silicon oxide relative to the weight of the α-alumina; and (c) from 0.01% w to 5% w, in particular 0.02% w to 3% w, of an alkali metal compound, calculated as the alkali metal oxide relative to the weight of the α-alumina.

In a preferred embodiment, the alumina carrier has an alumina content of at least 95% w and may be made by a method which comprises forming a mixture comprising: (a) from 65% w to 75% w, relative to the total weight of α-alumina in the mixture, of a first particulate α-alumina having a d₅₀ of from 10 μm to 60 μm, in particular from 12 μm to 40 μm; (b) from 25% w to 35% w, relative to the total weight of α-alumina in the mixture, of a second particulate α-alumina having a d₅₀ of from 2 μm to 6 μm; (c) from 2% w to 5% w of an alumina hydrate, calculated as aluminum oxide relative to the total weight of α-alumina in the mixture; (d) from 0.2% w to 5% w of an amorphous silica compound, as specified hereinbefore, calculated as silicon oxide relative to the total weight of α-alumina in the mixture; and (e) from 0.05% w to 0.3% w, of an alkali metal compound, calculated as the alkali metal oxide relative to the total weight of α-alumina in the mixture; and then forming the mixture into shaped bodies and firing the shaped bodies at a temperature of from 1050° C. to 1500° C. to form the carrier.

The preferred alumina hydrate is boehmite, though gibbsite, bayerite or diaspore may also be used.

Suitable alkali metals are, for example, lithium, sodium and potassium, or combination thereof. Suitable alkali metal compounds are, for example, alkali metal carbonates, alkali metal acetates, alkali metal formates, alkali metal nitrates, and combinations thereof. Typically, the overall atomic ratio of silicon to the alkali metal is in the range of from 1 to 10, more typically 2 to 8, for example 6. The overall atomic ratio of silicon to the alkali metal is deemed to relate to the total alkali metal content and the total silicon content of the carrier, which includes any alkali metal and any silicon which may be present in the carrier other than in the bond material.

It is also preferred that the carrier particles be prepared in the form of shaped bodies, the size of which is in general determined by the dimensions of a reactor in which they are to be deposited. Generally, however, it is found very convenient to use particles such as shaped bodies in the form of powdery particles, trapezoidal bodies, cylinders, saddles, spheres, doughnuts, and the like. The cylinders may be solid or hollow, straight or bent, and they may have the same length and cross-sectional dimensions which may be from 5 mm to 10 mm. Preferably, for use in a tubular fixed bed reactor, they are formed into a rounded shape, for example in the form of spheres, pellets, cylinders, rings or tablets, typically having dimensions in the range of from 2 mm to 2 cm.

The shaped bodies can be formed from the mixture by any convenient molding process, such as spraying, spray drying, agglomeration or pressing, but preferably they are formed by extrusion of the mixture. For applicable methods, reference may be made to, for example, U.S. Pat No. 5,145,824; U.S. Pat. No. 5,512,530; U.S. Pat. No. 5,384,302; U.S. Pat. No. 5,100,859; and U.S. Pat. No. 5,733,842, all of which are hereby incorporated by reference. To facilitate such molding processes, in particular extrusion, the mixture may suitably be compounded with up to 30% w and preferably from 2% w to 25% w, based on the weight of the mixture, of extrusion aids. Extrusion aids (also referred to by the term “processing aids”) are known in the art (cf., for example, “Kirk-Othmer Encyclopedia of Chemical Technology”, 4th edition, Volume 5, pp. 610 ff.). Suitable extrusion aids may be, for example, petroleum jelly, hydrogenated oil, synthetic alcohol, synthetic ester, glycol, polyolefin oxide or polyethylene glycol. Burnout materials are typically applied in a quantity of up to 30% w, in particular from 2% w to 25% w, relative to the weight of the mixture. Boric acid may also be added to the mixture, for example, in a quantity of up to 0.5% w, preferably in a quantity of from 0.01% w to 0.5 % w. The effect of the presence of boric acid may be a reduced content of leachable alkali metal ions in the carrier after firing. Enough water may be added to the mixture to make the mixture extrudable (by the term “the weight of the mixture”, as used hereinbefore, is meant the weight of the total mixture, but excluding the weight of any added water).

The shaped bodies are then dried and fired at a temperature high enough to ensure that the alumina particles are joined together by a sintering action and/or by the formation of bond posts formed from the bond material, if incorporated in the mixture. Generally, drying may take place between 0° C. and 400° C. and preferably between 30° C. and 300° C., typically for a period of up to 100 hours and preferably for from 5 minutes to 50 hours. Typically, drying is performed to the extent that the mixture contains less than 2% w of water. Generally, firing may take place between 1050° C. and 1500° C., typically between 1100° C. and 1470° C., preferably between 1150° C. and 1450° C., typically for a period of up to 5 hours and preferably for from 2 to 4 hours. Drying and firing may be carried out in any atmosphere, such as in air, nitrogen, or helium, or mixtures thereof. Preferably, in particular when the formed bodies contain organic material, the firing is at least in part or entirely carried out in an oxidizing atmosphere, such as an oxygen containing atmosphere. The terms “fired” and “calcined” may be used interchangeably as well as the terms “shaped” and “formed”.

It has been found that the performance of the catalyst may be enhanced if the carrier is washed, to remove soluble residues, before deposition of other catalyst ingredients on the carrier. On the other hand, unwashed carriers may also be used successfully. A useful method for washing the carrier comprises washing the carrier in a continuous fashion with hot, demineralized water, until the electrical conductivity of the effluent water does not further decrease. A suitable temperature of the demineralized water is in the range of 80° C. to 100° C., for example 90° C. or 95° C. Reference may be made to WO-00/15333, which is hereby incorporated by reference.

In a suitable method of catalyst preparation, the carrier is impregnated with a liquid composition of compounds of silver and potassium and rhenium or other useful additives, and subsequently dried by heating at a temperature in the range of from 150° C. to 500° C., preferably from 200° C. to 450° C., for a period of from 1 minute to 24 hours, preferably 2 minutes to 2 hours, and more preferably 2 minutes to 30 minutes, in an atmosphere of air, an inert gas, such as nitrogen or argon, or steam.

Reducing agents will generally be present to effect the reduction of a silver compound to metallic silver. For example, a reducing atmosphere, such as a hydrogen containing gas, may be employed, or a reducing agent may be present in one or more of the impregnation liquids, for example oxalate. If desired, the pore impregnation may be carried out in more than one impregnation and drying step. For example, silver may be impregnated in more than one step, and the promoters may be impregnated in one or more separate steps, prior to silver impregnation, after silver impregnation or intermediate to separate silver impregnation steps. The liquid composition is typically a solution, more typically an aqueous solution.

The compounds employed in the impregnation may independently be selected from, for example, inorganic and organic salts, hydroxides and complex compounds. They are employed in such a quantity that a catalyst is obtained of the desired composition.

The catalysts containing the supports of the present invention are useful for oxidation of any olefin which has at least three carbon atoms. Typically the number of carbon atoms is at most ten, more typically at most five. It is most preferred that the number of carbon atoms is three.

Apart from having an olefinic linkage (i.e. a moiety >C═C<), the olefin may comprise another olefinic linkage, or any other kind of unsaturation, for example in the form of an aryl group, for example a phenyl group. Thus, the olefin may be a conjugated or non-conjugated diene or a conjugated or non-conjugated vinyl aromatic compound, for example 1,3-butadiene, 1,7-octadiene, styrene or 1,5-cyclooctadiene.

In preferred embodiments, the olefin comprises a single olefinic linkage and for the remainder it is a saturated hydrocarbon. It may be linear, branched or cyclic. A single alkyl group may be attached to the olefinic linkage, such as in 1-hexene, or two alkyl groups may be attached to the olefinic linkage, such as in 2-methyl-octene-1 or pentene-2. It is also possible that three or four alkyl groups are attached to the olefinic linkage. Two alkyl groups may be linked together with a chemical bond, so that together with the olefinic linkage they form a ring structure, such as in cyclohexene. In these preferred embodiments, a hydrogen atom is attached to the olefinic linkage at the places which are not occupied by an alkyl group. It is particularly preferred that a single alkyl group is attached to the olefinic linkage.

The most preferred olefins having at least 3 carbon atoms are 1-pentene, 1-butene and, in particular, propylene. The skilled person will appreciate that in accordance with the geometry of its molecules, an olefin may yield a mixture of olefin oxides, for example olefin oxides in more than one isomeric form.

Generally, the process of this invention is carried out as a gas phase process, which is a process wherein gaseous reactants are reacted under the influence of a solid catalyst. Frequently, the reactants and any further components fed to the process are mixed to form a mixture which is subsequently contacted with the catalyst. The ratio of the quantities of the reactants and the further components, if any, and the further reaction conditions are not material to this invention and they may be chosen within wide ranges. As, generally, the mixture contacted with the catalyst is gaseous, the concentrations of the quantities of the reactants and the further components, if any, are specified below as a volume fraction of the mixture in gaseous form.

The concentration of the olefin may suitably be at least 0.1% v, typically at least 0.5% v, and the concentration may suitably be at most 60% v, in particular at most 50% v. Preferably, the concentration of the olefin is in the range of from 1% v to 40% v. If the olefin is propylene, 1-butene or 1-pentene it is preferred that its concentration is in the range of from 1% v to 30% v, preferably from 1.5% v to 20% v, and more preferably from 2% v to 15% v.

The concentration of oxygen may suitably be at least 2% v, typically at least 4% v, and in practice the concentration is frequently at most 20% v, in particular at most 15% v. If the olefin is propylene, 1-butene or 1-pentene it is preferred that the concentration of oxygen is in the range of from 6% v to 15% v, preferably 7% v to 15% v, and more preferably from 8% v to 15% v. The source of oxygen may be air,but it is preferred that an oxygen containing gas which may be obtained by separation from air is used.

Organic chloride compounds may be added to the mixture as a moderator of the catalyst, improving the selectivity. Examples of such organic chloride compounds are alkyl chlorides and alkenyl chlorides. Methyl chloride, vinyl chloride, 1,2-dichloroethane and in particular ethyl chloride are preferred organic chloride compounds. The organic chloride compounds may be used at a concentration of at least 0.1 ppm by volume, typically at least 0.2 ppm by volume, and preferably at least 1 ppm by. In particular, in the case of propylene, the organic chloride concentration should be at least 20 ppm by volume, more preferably at least 50 ppm by volume, and the concentration may be at most 2000 ppm by volume, in particular at most 1500 ppm by volume, wherein ppm by volume is calculated as the molar quantity of chlorine atoms in the total quantity of the reactant mixture.

The performance of the catalyst of the present invention may be improved by adding to the mixture a nitrate or nitrite-forming compound. A nitrate or nitrite-forming compound is meant to be a compound which is capable under the conditions at which it is contacted with the catalyst of introducing nitrate or nitrite ions on to the catalyst. In general, the nitrate or nitrite ions tend to disappear from the catalyst during the process, in which case they need to be replenished. As a consequence, it is preferred to add the nitrate or nitrite-forming compound continuously to the mixture, or in a discontinuous mode at least at the points in time that the need thereto arises. For the initial stage of the process it may be sufficient to add the nitrate or nitrite forming compound or nitrate or nitrite ions to the catalyst at the stage of catalyst preparation. Preferred nitrate or nitrite forming compounds are nitric oxide, nitrogen dioxide and/or dinitrogen tetraoxide. Alternatively, hydrazine, hydroxylamine, ammonia, nitromethane, nitropropane or other nitrogen containing compounds may be used. A mixture of nitrogen oxides is preferably used, which may be designated by the general formula NOₓ, wherein x is a number in the range of from 1 to 2, expressing the molar average atomic ratio of oxygen and nitrogen of the nitrogen oxides in the mixture.

In particular, for propylene oxidation, the nitrate or nitrite forming compound may suitably be used at a concentration of at least 10 ppm by volume, typically at least 50 ppm by volume, and the concentration may suitably be at most 500 ppm by volume, in particular at most 300 ppm by volume.

Carbon dioxide may or may not be present in the mixture. Carbon dioxide may reduce catalyst activity and selectivity and, thus, the yield of olefin oxide. Carbon dioxide may typically be present at a concentration of at most 35% v, in particular at most 20% v.

Furthermore, inert compounds may be present in the mixture, for example nitrogen or argon. In one embodiment of the present invention, methane is present in the mixture, as methane may improve the dissipation of the heat of reaction, without adversely effecting the selectivity and the conversion.

The process may preferably be carried out at a temperature of at least 150° C., in particular at least 200° C. Preferably, the temperature is at most 320° C., in particular, at most 300° C. The process may preferably be carried out at a pressure of at least 0.5 barg (i.e. bar gauge), in particular at least 1 barg. Preferably, the pressure is at most 100 barg, in particular at most 50 barg.

In general, it is preferred to operate at a high oxygen concentration. However, in actual practice in order to remain outside the flammability limits of the mixture of reactants and any further components present therein, the concentration of oxygen has to be lowered as the concentration of the olefin is increased. The actual safe operating conditions depends along with the gas composition, also on individual plant conditions, such as temperature and pressure, and tube sizes. When operating the process as a gas phase process using a packed bed reactor, the GHSV may preferably be at least 100 Nl/(l.h), in particular at least 200 Nl/(l.h). The GHSV may preferably be at most 30000 Nl/(l.h), in particular at most 15000 Nl/l.h). The term “GHSV” stands for the Gas Hourly Space Velocity, which is the volumetric flow rate of the feed gas, which is herein defined at normal conditions (i.e. 0° C. and 1 bar absolute), divided by the volume of the catalyst bed.

### EXAMPLES

### Example 1

### Preparation of Silver-Amine-Oxalate Stock Solution

A silver-amine-oxalate stock solution was prepared by the following procedure:

415 g of reagent-grade sodium hydroxide were dissolved in 2340 ml de-ionized water and the temperature was adjusted to 50° C. 1699 g high purity “Spectropure” silver nitrate was dissolved in 2100 ml de-ionized water and the temperature was adjusted to 50° C. The sodium hydroxide solution was added slowly to the silver nitrate solution, with stirring, while maintaining a solution temperature of 50° C. This mixture was stirred for 15 minutes, then the temperature was lowered to 40° C. Water was removed from the precipitate created in the mixing step and the conductivity of the water, which contained sodium and nitrate ions, was measured. An amount of fresh deionized water equal to the amount removed was added back to the silver solution. The solution was stirred for 15 minutes at 40° C. The process was repeated until the conductivity of the water removed was less than 90 μmho/cm. 1500 ml fresh deionized water was then added.

630 g of high-purity oxalic acid dihydrate were added in approximately 100 g increments. The temperature was kept at 40° C. and the pH was maintained at a level above 7.8. Water was removed from this mixture to leave a highly concentrated silver-containing slurry. The silver oxalate slurry was cooled to 30° C. Then 699 g of 92% w ethylenediamine (8% de-ionized water) was added while maintaining a temperature no greater than 30° C. The resulting solution contained approximately 27-33% w silver.

### Example 2

### Preparation of α-Alumina Carrier

An α-alumina carrier was made by mixing the following:

1. 67.4 parts by weight (pbw) of an α-alumina with d₅₀ of 29 μm;

2. 29 pbw of an α-alumina with d₅₀ of 3 μm;

3. 3 pbw of aluminium oxide (in the form of boehmite);

4. 0.5 pbw of silica (in the form of ammonia stabilized silica sol); and

5. 0.1 pbw of sodium oxide (in the form of sodium acetate).

To this mixture were added 5% w, relative to the mixture weight, of petroleum jelly and 9% w, relative to the mixture weight, of burnout material and 0.1% w, relative to the mixture weight, of boric acid. Water (30% w, relative to the mixture weight) was then added in an amount to make the mixture extrudable and this mixture was then extruded to form shaped bodies in the form of hollow cylinders that were 8 mm in diameter and 8 mm long. These were then dried and fired in a kiln at 1425° C. for 4 hours in air to produce the α-alumina carrier. The pore volume and the pore size distribution are measured by a conventional mercury intrusion device in which liquid mercury is forced into the pores of the carrier. Greater pressure is needed to force the mercury into the smaller pores and the measurement of pressure increments corresponds to volume increments in the pores penetrated and hence to the size of the pores in the incremental volume. The pore volume in the following description was determined by mercury intrusion under pressures increased by degrees to a pressure of 3.0×10⁸ Pa using a Micromeritics Autopore 9200 model (130° contact angle and mercury with a surface tension of 0.473 N/m). The resulting carrier had a surface area of 2.04 m² /g, a water absorption of 0.42 g/g and a pore volume of 0.41 ml/g.

### EXAMPLES 3-6

### Catalyst Preparation and T Sting for Propylene Oxide (PO)

The carrier as prepared in Example 2 was washed with water prior to its use in the preparation of the catalysts for Examples 3-6. In a typical carrier washing procedure a sample of carrier was placed in a stainless steel basket and submerged in hot 85° C. to 95° C. water. Water was passed over the carrier in a continuous manner and the conductivity of the wash was monitored. The carrier was considered sufficiently washed if no significant conductivity change occurred over 10 to 15 minutes. The carrier was then dried at 120° C. overnight. Reference may be made to WO-00/15333.

### Examples 3, 4

1.842 g of potassium nitrate were dissolved in 36.6 g of water. This solution was added to 240 g of silver stock solution prepared as above. Approximately 50 g of an α-alumina carrier as prepared in Example 2, was placed under a 25 mm Hg vacuum for 1 minute at ambient temperature. Approximately 130 g of the impregnating solution was then introduced in order to submerse the carrier, and the vacuum was maintained at 25 mm Hg for an additional 3 minutes. The vacuum was then released and the excess impregnating solution was removed from the catalyst pre-cursor by centrifugation at 500 rpm for two minutes. The catalyst pre-cursor was then dried while being shaken at 250° C. for 5.5 minutes in a stream of air. The catalyst precursor was allowed to cool down to room temperature and the impregnation was repeated the second time with the remainder of doped silver solution. Silver content of the resulting catalyst in Examples 3 and 4 was 25%, additional properties are listed in Table 1.

### Examples 5, 6

1.842 g of potassium nitrate and 0.6116 g of ammonium perrhenate were dissolved in 36.6 g of water. This solution was added to 240 g of silver stock solution prepared as above. Approximately 50 g of an α-alumina carrier prepared as in Example 2, was placed under a 25 mm Hg vacuum for 1 minute at ambient temperature. Approximately 130 g of the impregnating solution was then introduced in order to submerse the carrier, and the vacuum was maintained at 25 mm Hg for an additional 3 minutes. The vacuum was then released and the excess impregnating solution was removed from the catalyst pre-cursor by centrifugation at 500 rpm for two minutes. The catalyst pre-cursor was then dried while being shaken at 250° C. for 5.5 minutes in a stream of air. The catalyst precursor was allowed to cool down to room temperature and the impregnation was repeated the second time with the remainder of the doped silver solution. Silver content of the catalyst in Example 5 and 6 was 25%, additional properties are listed in Table 1.

Catalyst Testing

Catalyst testing was done in microreactors. Crushed pellets were sieved to 12-20 mesh and 15 gram of catalyst were loaded into a typical microreactor U-tube. The catalyst was tested at 45 psig, 600 GHSV, 8% propylene , 12% oxygen , 150 sccm gas flow, and 25 or 100 ppm NO₂. Nitrogen was used as a ballast gas. Ethyl chloride concentration was 150 ppm. Catalyst testing was done as follows: full feed composition was established at 200° C., then the catalysts were ramped at 10° C./hour to 230° C. and held there for 2 days at 100 ppm NOₓ. After that NOₓ was changed and catalyst was allowed to stabilize. Data at 25 ppm NOx was taken after 5 days on stream.

The results on catalyst performance, measured as the selectivity (Sel %) and the work rate (WKR) at the point in time that the selectivity had stabilised, are given in Table 1. The selectivity is calculated as the % mole of propylene oxide produced, relative to the propylene consumed. The work rate is the rate of propylene oxide production per unit weight of catalyst (kg/(kg.h)).

TABLE 1

| | | | | | % PO in |
|---|---|---|---|---|---|
| Example | | | NOx | WKR | the |
| No. | Formulation | Sel % | ppm | KgPO/m³/h | outlet |
| 18 | 80/μmol/g K | 43 | 100 | 7.8 | 0.52 |
| 19 | 80/μmol/g K | 50 | 25 | 8.2 | 0.54 |
| 20 | 80 μmol/g K, | 50.5 | 100 | 15.3 | 1.0 |
| | 10 μmol Re | | | | |
| 21 | 80 μmol/g K, | 55 | 25 | 10.7 | 0.69 |
| | 10 μmol Re | | | | |

The results in Table 1 indicate that the addition of ammonium perrhenate (NH₄ReO₄) to potassium promoted catalysts improves catalyst selectivity and productivity. Higher catalyst selectivity results in a more economical process for olefin oxide production. Higher selectivity leads to lower carbon dioxide emissions resulting in a more environmental friendly process. Higher catalyst productivity (yield) can result in lower operating temperature or construction of a smaller reactor resulting in substantial savings.

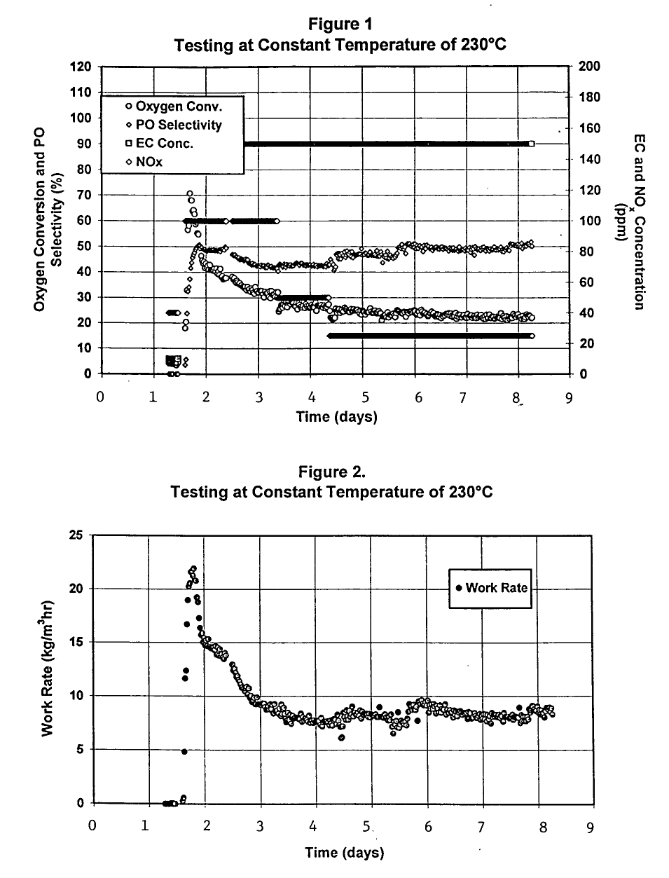

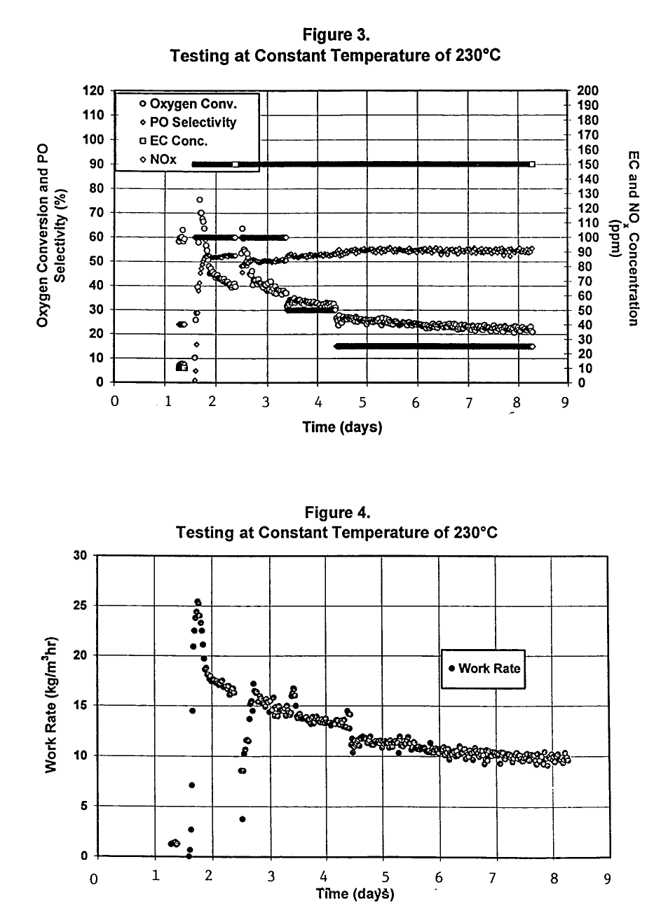

The performance of a potassium promoted silver catalyst as measured by selectivity and oxygen conversion is shown in FIG. 1. FIG. 2 illustrates the work rate for a potassium promoted silver catalyst. The performance of a rhenium containing potassium promoted silver catalyst as measured by selectivity and oxygen conversion is shown in FIG. 3. FIG. 4 illustrates the work rate for a rhenium containing potassium promoted silver catalyst.

Clearly, the addition of ammonium perrhenate (NH₄ReO₄) to potassium promoted propylene oxide catalysts provides an opportunity to improve catalyst selectivity and productivity.

The catalysts of the present invention are useful in a variety of catalytic applications in which a reactant stream (gaseous or liquid) is contacted with a catalyst supported on a carrier at elevated temperatures. There are many such processes in the chemical industry but the present carrier has proved itself particularly suitable in the catalytic formation of olefin oxide from a gas stream comprising propylene and oxygen. The utility of the present invention is however not so limited.

The instant application shows a detailed description of particular embodiments of the invention as described above. It is understood that all equivalent features are intended to be included within the claimed contents of this invention.

what is claimed is:

1. A process for the oxidation of an olefin comprising three or more carbon atoms, wherein the process comprises:
reacting the olefin with oxygen to form a reaction mixture in the presence of a catalyst composition comprising:
silver; and,
a promoter comprising potassium and a promoter comprising rhenium deposited on an α-alumina carrier, wherein the potassium promoter provides potassium at a concentration of up to 120 μmole per gram of catalyst composition.

2. The process of claim 1, wherein the potassium promoter provides potassium at a concentration of from 12 μmole to 100 μmole per gram of catalyst composition and the rhenium promoter provides rhenium at a concentration of from 3 μmole to 20 μmole per gram of catalyst composition.

3. The process of claim 2, wherein the α-alumina carrier has a BET surface area of 0.1 m²/g to 25 m²/g, and an apparent porosity of from 0.1 ml/g to 1.2 ml/g.

4. The process of claim 1, wherein the α-alumina carrier comprises at least 60% w α-alumina.

5. The process of claim 1, wherein the α-alumina carrier has a pore size distribution such that the pores with diameters in the range of from 0.2 μm to 10 μm comprise more than 75% of the total pore volume; the pores with diameters greater than 10 μm comprise less than 20% of the total pore volume; and the pores with diameters less than 0.2 1m comprise less than 10% of the total pore volume.

6. The process of claim 1, wherein the α-alumina carrier has a water absorption of at least 0.35 ml/g and a surface area in the range of from 1.0 m²/g to 5 m2/g.

7. The process of claim 1, wherein the α-alumina carrier is based on:
(a) from 50% w to 90% w of a first particulate α-alumina having an average particle size of from more than 10 μm up to 100 μm; and,
(b) from 10% w to 50% w of a second particulate α-alumina having an average particle size of from 1 μm to 10 μm; said % w being based on the total weight of α-alumina in the mixture.

8. The process of claim 1, wherein the α-alumina carrier comprises:
(a) from 65% w to 75% w, relative to the total weight of α-alumina in the mixture, of a first particulate α-alumina having an average particle size of from 11 μm to 60 μm;
(b) from 25% w to 35% w, relative to the total weight of α-alumina in the mixture, of a second particulate α-alumina having an average particle size of from 2 μm to 6 μm;
(c) from 2% w to 5% w of an alumina hydrate, calculated as aluminum oxide relative to the total weight of α-alumina in the mixture;
(d) from 0.2% w to 0.8% w of an amorphous silica compound, calculated as silicium oxide relative to the total weight of α-alumina in the mixture; and, (e) from 0.05% w to 0.3% w of an alkali metal compound, calculated as the alkali metal oxide relative to the total weight of α-alumina in the mixture.

9. The process of claim 1 wherein the reaction mixture further comprises an organic chloride promoter.

10. The process of claim 9 wherein the organic chloride is present at a concentration of at least 50 ppm by volume.

11. The process of claim 9, wherein the reaction mixture further comprises a NO, promoter, wherein x is 1 or 2.

12. The process of claim 9, wherein the NOₓ promoter is present at a concentration of at least 10 ppm by volume.

13. A catalyst composition for the oxidation of an olefin comprising three or more carbon atoms, wherein the catalyst composition comprises:
silver; and,
a promoter comprising potassium and a promoter comprising rhenium deposited on an α-alumina carrier, wherein the potassium promoter provides potassium at a concentration of from 8 μmole to 120 μmole per gram of catalyst composition.

14. The catalyst of claim 13, wherein the rhenium promoter provides rhenium at a concentration of from 1 μmole to 30 μmole per gram of catalyst composition.

15. The catalyst of claim 13, wherein the carrier comprises an α-alumina carrier is based on:
(a) from 50% w to 90% w of a first particulate α-alumina having an average particle size of from more than 10 up to 100 μm; and,
(b) from about 10% w to about 50% w of a second particulate α-alumina having an average particle size of from 1 μm to 10 μm; and wherein said % w is based on the total weight of α-alumina in the mixture.

16. The catalyst of claim 13, wherein α-alumina carrier has a pore size distribution such that pores with diameters in the range of from 0.2 μm to 10 μm represent more than 75% of the total pore volume; pores with diameters greater than 10 μm represent less than 20% of the total pore volume; and pores with diameters less than 0.2 μm represent less than 10% of the total pore volume.

17. The catalyst composition of claim 13, wherein the α-alumina carrier has a water absorption of at least 0.35 ml/g and a surface area in the range of from 0.6 m²/g to 5 m²/g.

18. The catalyst of claim 13, wherein the carrier comprises an α-alumina carrier having a composition comprising:
(a) from 65% w to 75% w, relative to the total weight of α-alumina in the mixture, of a first particulate α-alumina having an average particle size of from 11 μm to 60 μm;
(b) from 25% w to 35% w, relative to the total weight of α-alumina in the mixture, of a second particulate α-alumina having an average particle size of from 2 μm to 6 μm;
(c) from 2% w to 5% w of an alumina hydrate, calculated as aluminum oxide relative to the total weight of α-alumina in the mixture;
(d) from 0.2% w to 0.8% w of an amorphous silica compound, calculated as silicium oxide relative to the total weight of α-alumina in the mixture; and
(e) from 0.05 to 0.3% w of an alkali metal compound, calculated as the alkali metal oxide relative to the total weight of α-alumina in the mixture.
